SEQUENCE LISTING (6 sequences)

SEQ ID NO: 1 - DNA, 708 bp, Oryza sativa
atggcgcccagagcagctacggtggagaaggttgctgtggcgccacccaccgggcttggtcttggcgtcggcggaggtgtcggagccgggggtcctcactacaggggcgtccgcaagcgcccgtgggggcgttacgcagcggagatccgtgaccctgccaagaagagccgggtgtggctcggtacctacgacacggcagaggaggccgcccgcgcctacgacgccgccgctcgagagttccggggtgccaaggcaaaaacaaactttccgtttgcatcacagtcgatggtcggctgtggcggcagccccagcagcaatagcacggtagacaccggtggcggcggggttcagacgcctatgcgggccatgcctctgccgccgactctggacttggatttgttccaccgcgcggctgctgtgactgcagtcgccggcaccggcgttcgctttcctttcagaggatatcccgttgcacgtccagcaacgcatccttactttttctatgagcaggctgcagcggctgccgcagctgaggctggataccgtatgatgaagcttgcaccgccggtcaccgtggcggcggttgcacaaagtgactccgactcctcgtcggtggttgatctcgcgccgtcacctccagcggttacggcgaacaaggcggcagctttcgatctggatctgaaccggccgccgccggtagagaactag

SEQ ID NO: 2 - protein, 235 aa, Oryza sativa
MAPRAATVEKVAVAPPTGLGLGVGGGVGAGGPHYRGVRKRPWGRYAAEIRDPAKKSRVWLGTYDTAEEAARAYDAAAREFRGAKAKTNFPFASQSMVGCGGSPSSNSTVDTGGGGVQTPMRAMPLPPTLDLDLFHRAAAVTAVAGTGVRFPFRGYPVARPATHPYFFYEQAAAAAAAEAGYRMMKLAPPVTVAAVAQSDSDSSSVVDLAPSPPAVTANKAAAFDLDLNRPPPVEN

SEQ ID NO: 3 - DNA, 20 bp, Artificial
  AP37 forward primer
atggcgcccagagcagctac

SEQ ID NO: 4 - DNA, 20 bp, Artificial
  AP37 reverse primer
ctagttctctaccggcggcg

SEQ ID NO: 5 - DNA, 20 bp, Artificial
  AP59 forward primer
atgctgcttaatccggcgtc

SEQ ID NO: 6 - DNA, 20 bp, Artificial
  AP59 reverse primer
ttagctcaccagctgctgga

(19) United States
(12) Patent Application Publication
(10) Pub. No.: US 2012/0144521 A1
(43) Pub. Date: Jun. 7, 2012

(54) TRANSCRIPTION FACTOR AP37 INCREASING RESISTANCE OF PLANTS AGAINST DROUGHT STRESS, AND USE THEREOF

(75) Inventors: Ju Kon Kim, Gyeonggi-do (KR); Se Jun Oh, Seoul (KR); Youn Shic Kim, Gyeonggi-do (KR)

(73) Assignee: Myongji University Industry and Academia Cooperation, Gyeonggi-do (KR)

(21) Appl. No.: 13/322,055

(22) PCT Filed: May 22, 2009
(86) PCT No.: PCT/KR09/02703

§ 371 Date: Nov. 22, 2011

(51) Int. Cl. A01H 5/00; A01H 1/00; C12N 15/82; C12N 5/10; C07K 14/415; C12N 15/29

(52) U.S. Cl. 800/278; 530/372; 536/23.6; 435/320.1; 435/419; 435/468; 800/298; 800/320.2

(57) ABSTRACT

The present invention is directed to transcription factor protein AP37 (APETELA 37) originating from rice which is related to tolerance of a plant to stress, a gene which encodes the protein, a recombinant vector comprising the gene, a host cell transformed with the recombinant vector, a method of producing a transgenic plant by using the recombinant vector, a method of improving tolerance of a plant to stress or increasing grain yield by transforming a plant cell with the recombination vector, a plant having improved tolerance to stress or increased grain yield produced according to said method, and a composition for improving tolerance of a plant to stress or for increasing grain yield under drought condition comprising the gene encoding AP37 protein as an effective component.

### TECHNICAL FIELD

The present invention relates to transcription factor AP37 originating from rice which can be used for improving tolerance of a plant to drought stress and increasing grain yield under drought condition and use of the same.

### BACKGROUND ART

Drought stress is among the most serious challenges to crop production worldwide. Upon exposure of plants to drought conditions, many stress-related genes are induced and their products are thought to function as cellular protectants of stress-induced damage. The expression of stress-related genes is largely regulated by specific transcription factors. Members of the AP2, bZIP, zinc finger, and MYB families have been shown to have regulatory roles in stress responses. The rice and Arabidopsis genomes code for more than 1300 transcriptional regulators, accounting for about 6% of the estimated total number of genes in both cases. About 45% of these transcription factors were reported to be from plant-specific families (Riechmann et al., (2000) Science 290: 2105-2110). One example of such a plant-specific family of transcription factors is APETALA2 (AP2), whose members share a highly conserved DNA-binding domain known as AP2.

AP2 factors appear to be widespread in plants with the genomes of rice and Arabidopsis predicted to contain 139 and 122 AP2 genes, respectively. Members of the AP2 family have been implicated in diverse functions in cellular processes involving flower development, spikelet meristem determinacy, plant growth, and stress tolerance. Of these diverse functions, the involvement of the AP2 family in stress response has been relatively well characterized. In particular, CBF/DREBs genes from Arabidopsis have been shown to play crucial roles in response to low temperature, salt, and drought stresses in transgenic Arabidopsis. CBF/DREBs are members of the AP2 family and identifiable by the presence of CBF/DREB signature motifs (PKK/RPAGRxKFxETRHP and DSAWR) directly flanking the AP2 domain. Overexpression of CBF/DREBs in transgenic Arabidopsis increases the transcript levels of stress-related genes and enhances tolerance to drought, high salinity and freezing stresses. CBF/DREBs are also heterologously effective in canola, tomato, tobacco and rice, enhancing stress tolerance in the corresponding transgenic plants. CBF/DREB orthologs have also been identified in canola, tomato, wheat, rye, barley and rice, and all of them are inducible by low temperature treatments. The AP2 gene family from other plant species, including DBF1 and DBF2 from maize, AhDREB1 from Atriplex hortensis, OPBP1 from tobacco, CaPF1 from pepper (Capsicum annuum), HvRAF from barley, and SodERF3 from sugarcane, have been found to be involved in responses to various abiotic stress conditions.

Approximately 20% of rice growing areas worldwide are prone to drought. Although drought conditions can alter the growth and development of rice at any time during its life cycle, drought stress during reproductive growth directly results in a loss of grain yield. To evaluate improvements in grain yield under drought conditions, it is important to subject the plants to the stress during the transition to their reproductive phase. To date, a number of studies have suggested that overexpression of stress related genes could improve drought tolerance in rice to some extent (Xu et al., (1996) Plant Physiol 110: 249-257; Garg et al., (2002) Proc Natl Acad Sci USA 99: 15898-15903). Despite such efforts to develop drought-tolerant rice plants, very few of these have been shown to improve grain yields under field conditions. Examples of positive effects include transgenic rice plants expressing SNAC1 (Hu et al., (2006) Proc Natl Acad Sci USA 103: 12987-12992) and OsLEA3 (Xiao at al., (2007) Theor Appl Genet 115: 35-46) which was shown to improve grain yield under field drought conditions.

### DISCLOSURE OF INVENTION

### Technical Problem

The present invention is devised in view of the above-described needs. Specifically, inventors of the present invention identified AP37 gene encoding transcription factors with the AP2 domain that are stress-inducible. Overexpression of the gene in transgenic rice contributed to the improvement of tolerance of a plant to drought, low temperature and high salinity at the vegetative stage. Further, these AP37 overexpressors showed significantly enhanced drought tolerance in the field, and increased grain yield by 16-57% over the control under severe drought conditions. As a result, the present invention was completed.

### Technical Solution

In order to solve the problems described above, the present invention provides transcription factor protein AP37 (APETELA 37) originating from rice which is related to tolerance of a plant to stress.

Further, the present invention provides a gene which encodes the protein and a recombinant vector comprising the gene.

Further, the present invention provides a host cell transformed with the recombinant vector.

Further, the present invention provides a method of producing a transgenic plant by using the recombinant vector.

Further, the present invention provides a method of improving tolerance of a plant to stress or increasing grain yield by transforming a plant cell with the recombination vector.

Further, the present invention provides a plant having improved tolerance to stress or increased grain yield, that is produced according to the method described above.

Still further, the present invention provides a composition comprising the gene encoding AP37 protein as an effective component, for improving tolerance of a plant to stress or for increasing grain yield under drought condition.

### Advantageous Effects

According to the present invention, yield of grain crops that are cultivated in dry regions, in particular rice, can be improved by using the transcription factor AP37 of the present invention. In addition, it can be advantageously used for increasing harvest amount of the crops that are cultivated in low temperature regions.

### BRIEF DESCRIPTION OF DRAWINGS

FIG. 1 shows alignments of AP2 domain sequences from 29 stress-inducible rice genes. Deduced amino acid sequences of the AP2 domains of the 29 genes listed in Table 1 were aligned using the CLUSTAL W program. Identical and conserved residues are highlighted (grey). Signature motifs are indicated by boxes with marks on the top as follows: Ia-c, IIa-c, IIIa-d, IVa, Va-c, and VIa-d for subgroups I-VI, respectively.

FIG. 2 shows expression of AP37 and AP59 in response to stress conditions in rice. Ten μg of total RNA was prepared from leaf tissues of 14 d-old seedlings exposed to drought, high salinity, ABA or low temperature for indicated time points: for drought stress, the seedlings were air-dried at 28° C.; for high-salinity stress, seedlings were exposed to 400 mM NaCl at 28° C.; for low-temperature stress, seedlings were exposed to 4° C. for ABA, seedlings were exposed to a solution containing 100 μM ABA. Total RNAs were blotted and hybridized with the AP37 and AP59 gene-specific probes. The blots were then reprobed for the Dip1 (Oh et al., (2005) Plant Physiol 138: 341-351) and rbcS (Jang et al., (1999) Mol Breeding 5: 453-461) genes which were used as markers for up- and down-regulation of key genes following stress treatments, respectively. EtBr staining was used to determine equal loading of RNAs.

FIGS. 3 to 5 show production of OsCc1:AP37 and OsCc1:AP59 transgenic rice plants.

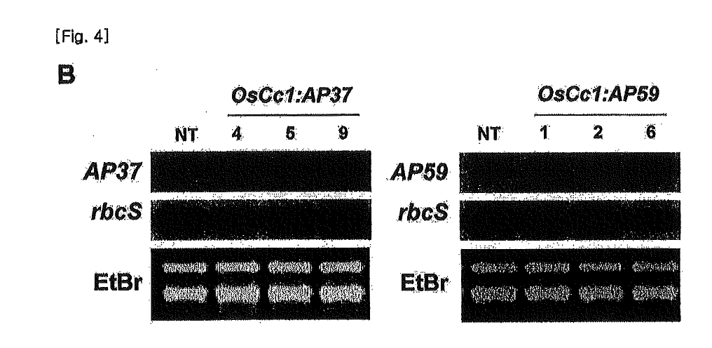

A (FIG. 3). The OsCc1:AP37 and OsCc1:AP59 plasmids consist of OsCc1 promoter (Jang et al., (2002). Plant Phisol 29: 1473-1481), a constitutive promoter linked to the AP37 and AP59 coding region, respectively, the 3′-region of the potato proteinase inhibitor II gene (3′PinII), and a gene expression cassette that contains the 35S promoter, the bar coding region, and the 3′-region of the nopaline synthase gene (3 nos). The entire expression cassette is flanked by the 5′-matrix attachment region (MAR) of the chicken lysozyme gene (Oh et al., (2005) Plant Cell Rep 4: 145-154).

B (FIG. 4), RNA gel-blot analysis was performed using total RNAs from young leaves of three homozygous T₄ lines of OsCc1:AP37 and OsCc1:AP59 plants, respectively, and of nontransgenic (NT) control plants. The blots were hybridized with the AP37 and AP59 gene specific probes, and reprobed for rbcS. EtBr staining was used to determine equal loading of RNAs.

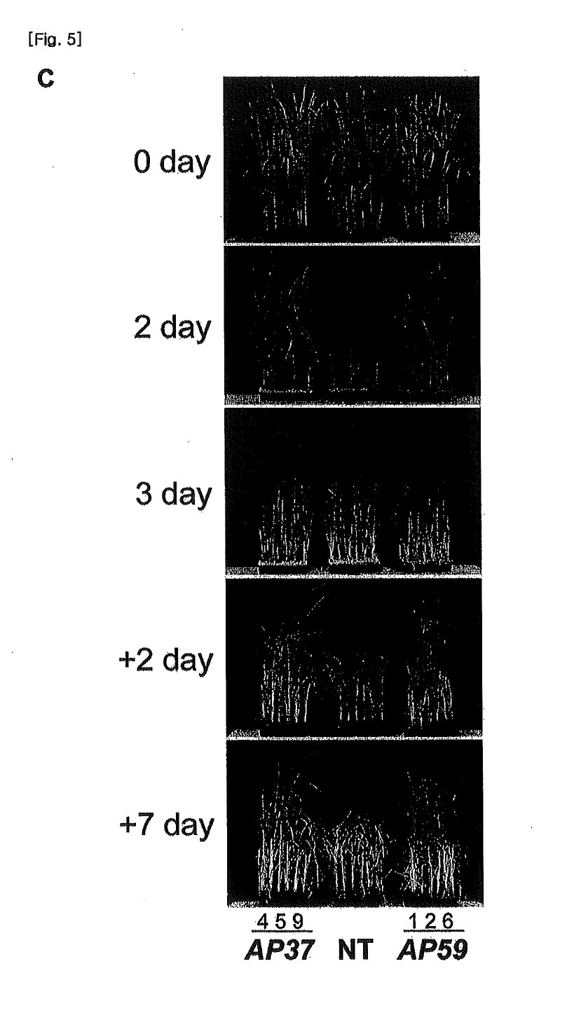

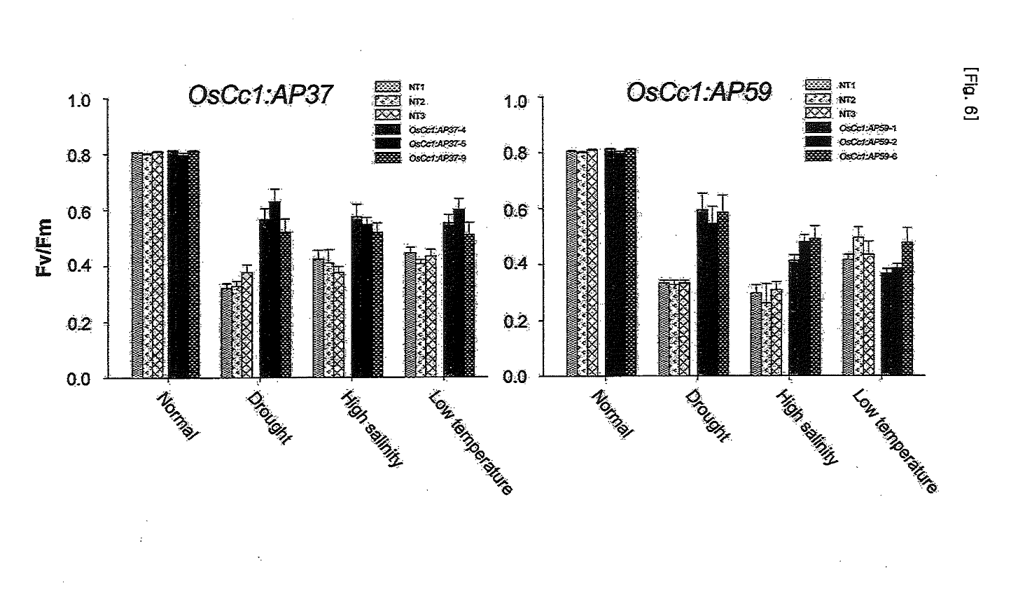

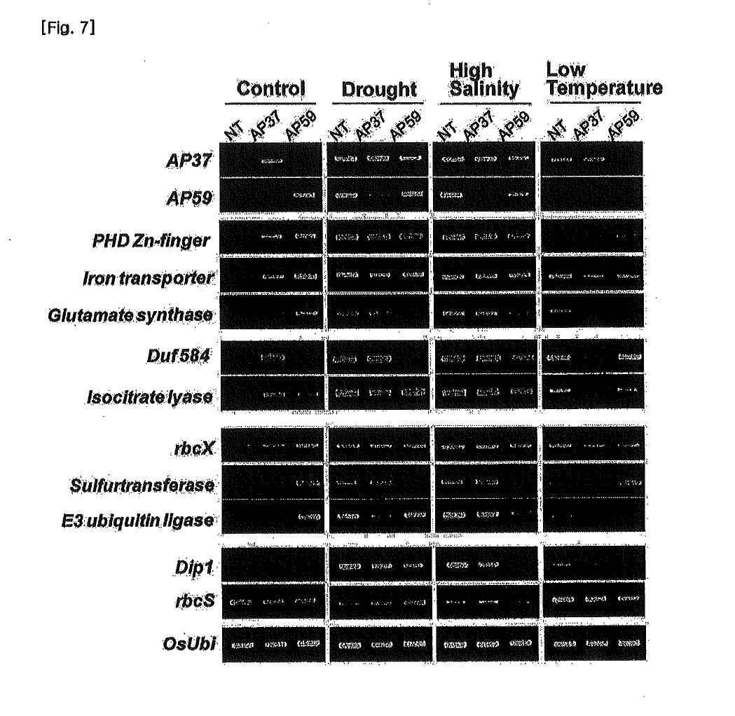

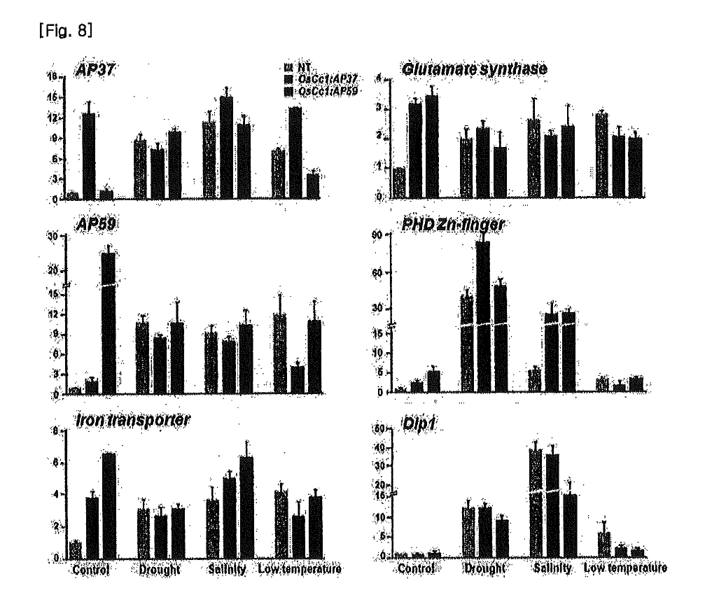

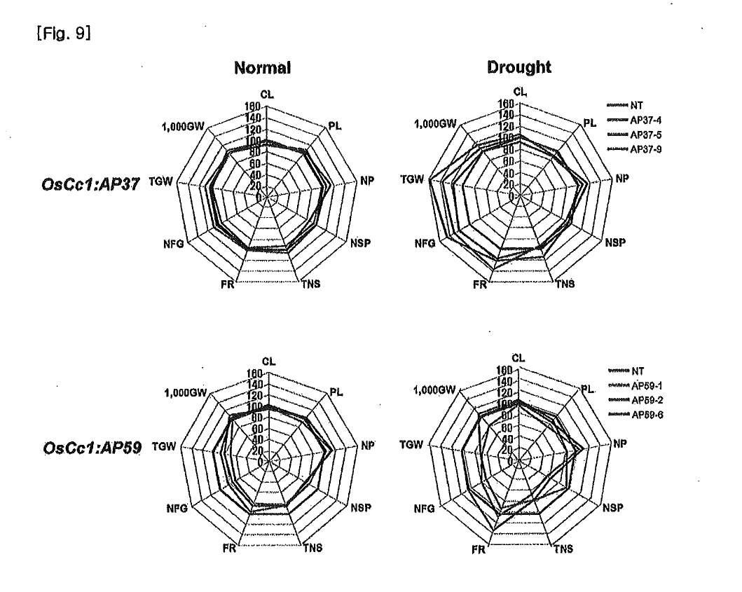

C (FIG. 5), Appearance of transgenic plants during drought stress.

Three independent homozygous T₄ lines of OsCc1:AP37 and OsCc1:AP59 plants and nontransgenic (NT) controls were grown in a greenhouse for 4 weeks and subjected to drought stress treatments. Four-week-old transgenic and NT plants were subjected to 3 d of drought stress, followed by 2 d and 7 d re-watering in the greenhouse, respectively. Images were taken at the indicated time points; ‘+’ denotes the number of re-watering days under normal growth conditions.

FIG. 6 shows changes in chlorophyll fluorescence (Fv/Fm) of rice plants under drought, high salinity and low temperature stress conditions. Three independent homozygous T₄ lines of OsCc1:AP37 and OsCc1:AP59 plants and nontransgenic (NT) controls grown in MS medium for 14 d were subjected to various stress conditions as described in the methods section. After stress treatments, the Fv/Fm values were measured using a pulse modulation fluorometer (mini-PAM, Waize, Germany). All plants were grown under a continuous light of 150 μmol m⁻² g⁻¹ prior to stress treatments. Each data point represents the mean±SE of triplicate determinations (n=10).

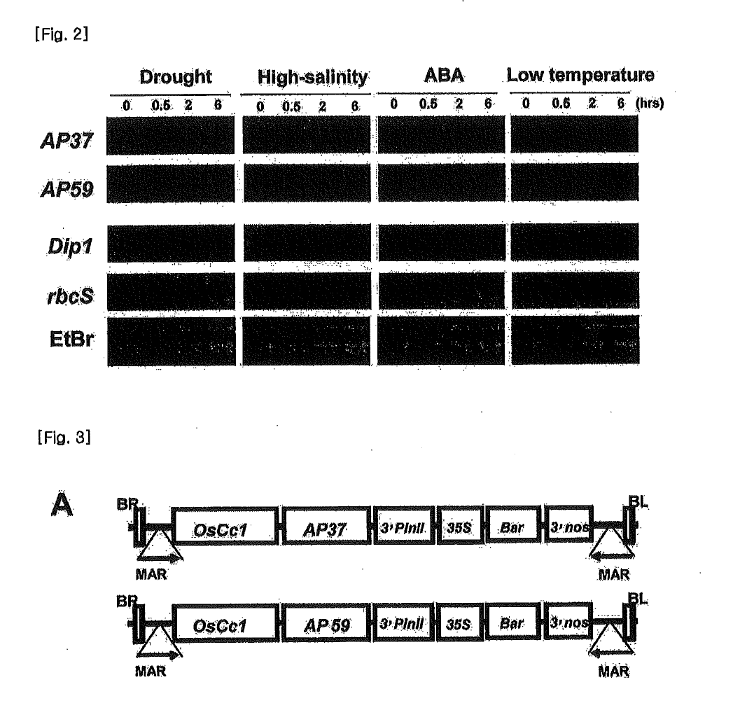

FIGS. 7 and 8 show regulated expression of stress-related genes in OsCc1:AP37, OsCc1:AP59, and nontransgenic (NT) plants under normal and stress conditions. Homozygous T₄ lines of OsCc1:AP37, OsCc1:AP59, and nontransgenic (NT) control rice plants were grown in a greenhouse for 14 d. Transgenic and NT plants were then treated with various stress conditions as described in the legends of FIG. 2. A (FIG. 7). Transcript levels of the AP37, AP59 and eight target genes were determined by RT-PCR. B (FIG. 8). Transcript levels of the AP37, AP59 and three target genes were determined by qRT-PCR, Dipl (Oh et al., (2005) Plant Physiol 138: 341-351) was used as a marker for up-regulation of key genes following stress treatments. The rice ubiquitin gene (OsUbi) was used as an equal loading control.

FIG. 9 shows agronomic traits for OsCc1:AP37 and OsCc1:AP59 rice plants grown in the field under normal and stress conditions. Spider plots of agronomic traits of three independent homozygous T₅ lines of OsCc1:AP37 and OsCc1:AP59, and corresponding nontransgenic (NT) controls under normal and drought conditions, respectively, were drawn by using the Microsoft Excel software. Each data point represents percentage of the mean values (n=20) listed in Tables 2 and 3. Mean values from NT controls were set at 100% as a reference, CL, culm length; PL, panicle length; NP, number of panicles per hill; NSP, number of spikelets per panicle; TNS, total number of spikelets; FR, filling rate; NFG, number of filled grains; TGW, total grain weight; 1,000 GW, thousand grain weight.

### MODE FOR THE INVENTION

In order to achieve the purpose of the invention as described above, the present invention provides transcription factor protein AP37 (APETELA 37) originating from rice which consists of the amino acid sequence of SEQ ID NO: 2.

The scope of the AP37 protein of the present invention includes a protein having an amino acid sequence represented by SEQ ID NO: 2 that is isolated from rice, and functional equivalents of said proteins. The term functional equivalent means that, as a result of addition, substitution or deletion of amino acid residues, it has an amino acid sequence with at least 70%, preferably at least 80%, more preferably at least 90%, still more preferably at least 95% homology with the amino acid sequence that is represented by SEQ ID NO: 2, thus indicating a protein which has substantially the same physiological activity as the protein expressed by SEQ ID NO: 2.

Further, the present invention provides a gene which encodes said AP37 protein. The gene of the present invention includes both genomic DNA and cDNA which encode AP37 protein. Preferably, the gene of the present invention may comprise a nucleotide sequence that is described by SEQ ID NO: 1. Variants of said nucleotide sequence are also within the scope of the present invention. Specifically, said gene may comprise a nucleotide sequence with at least 70%, preferably at least 80%, more preferably at least 90%, still more preferably at least 95% homology with the nucleotide sequences of SEQ ID NO: 1. The “sequence homology %” for a certain polynucleotide is determined by comparing two nucleotide sequences that are optimally arranged with a region to be compared. In this regard, a part of the polynucleotide sequence in a region to be compared may comprise an addition or a deletion (i.e., a gap) compared to a reference sequence (without any addition or deletion) relative to the optimized arrangement of the two sequences.

Further, the present invention provides a recombinant vector comprising the AP37 gene according to the present invention. Preferably, the recombinant vector is a recombinant plant expression vector.

The term “recombinant” indicates a cell which replicates a heterogeneous nucleotide or expresses said nucleotide, a peptide, a heterogeneous peptide, or a protein encoded by a heterogeneous nucleotide. Recombinant cell can express a gene or a gene fragment in a form of a sense or antisense, that are not found in natural state of cell. In addition, a recombinant cell can express a gene that is found in natural state, provided that said gene is modified and re-introduced into the cell by an artificial means.

The term “vector” is used herein to refer DNA fragment(s) and nucleotide molecules that are delivered to a cell. Vector can replicate DNA and be independently reproduced in a host cell. The terms “delivery system” and “vector” are often interchangeably used. The term “expression vector” means a recombinant DNA molecule comprising a desired coding sequence and other appropriate nucleotide sequences that are essential for the expression of the operatively-linked coding sequence in a specific host organism.

The vector of the present invention can be constructed as a vector which is typically used for cloning or expression. In addition, the vector of the present invention can be constructed by having a prokaryotic cell or an eukaryotic cell as a host. For example, when the vector of the present invention is an expression vector and a prokaryotic cell is employed as a host, a strong promoter for the initiation of transcription (e.g., pLλ promoter, trp promoter, lac promoter, T7 promoter, tac promoter and the like), and a ribosome binding site for the initiation of translation and a termination sequence for transcription/translation are generally comprised. When E. coli is employed as a host cell, a promoter and an operator region relating to the biosynthetic pathway of tryptophan in E. coli, and left-side promoter of phage λ(i.e., pLλpromoter) can be used as a regulation site.

Meanwhile, the vector which can be used for the present invention can be constructed by manipulating a plasmid (e.g., pSC101, ColE1, pBR322, pUC8/9, pHC79, pGEX series, pET series, pUC19 and the like), a phage (e.g., λgt4-λB, λ-Charon, λΔz1, M13 and the like) or a virus (e.g., SV40 and) that are often used in the pertinent art.

On the other hand, when the vector of the present invention is an expression vector and an eukaryotic cell is employed as a host, a promoter originating from genome of mammalian cell (e.g., metallothionein promoter) or a promoter originating from mammalian virus (e.g., adenovirus late promoter, vaccinia virus 7.5K promoter, SV40 promoter, cytomegalovirus promoter and tk promoter of HSV) can be utilized. As a termination sequence for transcription, a polyadenylation sequence is generally comprised.

The vector of the present invention may comprise as a selection marker an antibiotics-resistant gene that is conventionally used in the pertinent art, and the example includes a gene which is resistant to ampicillin, gentamycin, carbenicillin, chloramphenicol, streptomycin, kanamycin, geneticin, neomycin and tetrcycline.

Further, the present invention provides a host cell that is transformed with the recombinant vector of the present invention. Any kind of a host cell known in the pertinent art can be used if stable and continuous cloning and expression of the vector of the present invention can be achieved by using it. Examples include strains belonging to the genus Bascillus such as E. coli JM109, E. coil BL21, E. coli RR1, E. coli LE392, E. coli B, E. coli X 1776, E. coli W3110, Bascillus subtilus, Bascillus thuringiensis, and the like, Salmonella typhimurium, intestinal flora and strains such as Serratia marcescens and various Pseudomonas Spp. and the like.

In addition, when the vector of the present invention is transformed in an eukaryotic cell, a host cell such as Saccharomyce cerevisiae, an insect cell, a human cell (e.g., CHO cell line (Chinese hamster ovary), W138, BHK, COS-7, 293, HepG2, 3T3, RIN and MDCK cell line), a plant cell line and the like can be used. Preferably, the host cell is a plant cell.

When a host cell is a prokaryotic cell, transfer of the vector of the present invention into a host cell can be carried out according to CaCl₂ method, Hanahan's method (Hanahan, D., J. Mol. Biol., 166:557-580 (1983)), and an electroporation method, etc. In addition, when a host cell is an eukaryotic cell, the vector of the present invention can be transferred into a host cell according to a microscopic injection method, calcium phosphate precipitation method, an electroporation method, a liposome-mediated transformation, DEAE-dextran treatment method and a gene bombardment method, etc.

Further, the present invention provides a method of producing a transgenic plant, comprising steps of transforming a plant cell with the recombinant vector of the present invention and regenerating a transgenic plant from the obtained transformed plant cell.

The method described in the above comprises a step of transforming a plant cell with the recombinant vector of the present invention and the transformation can be mediated by Agrobacterium tumefaciens. In addition, the method described in the above comprises a step of regenerating a transgenic plant from the transformed plant cell. Any method that is known in the pertinent art as a method of regenerating a transgenic plant from a transformed plant cell can be used for the present invention.

Further, the present invention provides a method of improving tolerance of a plant to stress comprising a step of transforming a plant cell with the recombinant vector of the present invention to overexpress the AP37 gene in the plant. The stress can be preferably drought, high salinity, or low temperature stress. Most preferably, it can be drought stress. The high salinity condition can be, for example, 400 mM NaCl, and the low temperature condition can be 4° C., for example.

Further, the present invention provides a plant having improved tolerance to stress, that is produced according to the method described above. The plant can be preferably a monocot plant including rice, barley, maize, wheat, rye, sugar cane, oat, onion and the like. More preferably, it is rice.

Further, the present invention provides a method of improving increasing grain yield comprising steps of transforming a plant cell with the recombinant vector of the present invention and overexpressing the AP37 gene in the plant under drought condition. According to this method, grain yield was increased by 16 to 57% under drought condition compared to a control group.

Further, the present invention provides a plant having increased grain yield that is produced according to the method described above. Herein, the plant can be preferably a monocot plant including rice, barley, maize, wheat, rye, sugar cane, oat, onion and the like. More preferably, it is rice.

Further, the present invention provides a composition for improving tolerance of a plant to stress comprising as an effective component the gene encoding AP37 protein, which consists of an amino acid sequence of SEQ ID NO: 2. Since the AP37 gene of the present invention has an activity of improving tolerance of a plant to stress, a composition comprising this gene can be advantageously used for improving tolerance of a plant to stress. The stress can be preferably drought, high salinity, or low temperature stress. Most preferably, it is drought stress.

Still further, the present invention provides a composition for increasing grain yield under drought condition comprising as an effective component the gene encoding AP37 protein, which consists of an amino acid sequence of SEQ ID NO: 2.

The present invention will now be described in greater detail with reference to the following examples. However, it is only to specifically exemplify the present invention and in no case the scope of the present invention is limited by these examples.

Materials and Methods

Plasmid Construction and Transformation of Rice

The expression plasmids OsCc1:AP37 and OsCc1:AP59 contained the bar gene under the control of the cauliflower mosaic virus 35S promoter to enable herbicide-based plant selection. The OsCc1 promoter was used to drive constitutive plasmid gene expression. The coding regions of AP37 and AP59 were amplified from rice total RNA using an RT-PCR system (Promega, Wis.), according to the manufacturer's instructions. Primer pairs were as follows: AP37, forward (5′-ATGGCGCCCAGAGCAGCTAC-3′; SEQ ID NO: 3) and reverse (5′-CTAGTTCTCTACCGGCGGCG-3′; SEQ ID NO: 4); AP59, forward (5′-ATGCTGCTTAATCCGGCGTC-3′; SEQ ID NO: 5) and reverse (5′-TTAGCTCACCAGCTOCTGGA-3′; SEQ ID NO: 6). Plasmids were introduced into Agrobacterium tumefaciens LBA4404 by triparental mating and embryogenic (Oryza sativa cv Nakdong) calli from mature seeds were transformed as previously described (Jang et al., (1999) Mol Breeding 5: 453-461).

Drought Treatments at Vegetative Stage

Transgenic and nontransgenic (NT) rice (Oryza sativa cv Nakdong) seeds were germinated in half-strength MS solid medium in a growth chamber in the dark at 28° C. for 4 d, transplanted into soil and then grown in a greenhouse (16-h-light/8-h-dark cycles) at 28-30° C. Eighteen seedlings from each transgenic and nontransgenic line were grown in pots (3×3×5 cm; 1 plant per pot) for 4 weeks before undertaking the drought-stress experiments. To induce drought stress, 4-week-old transgenic and NT seedlings were unwatered for 3 d followed by 7 d of watering. The numbers of plants that survived or continued to grow were then scored.

Chlorophyll Fluorescence Under Conditions of Drought, High-Salinity and Low Temperature

Transgenic and nontransgenic rice (Oryza sativa cv Nakdong) seeds were germinated and grown in half-strength MS solid medium for 14 d in a growth chamber (16-h-light of 150 μmol m⁻²s⁻¹/8-h-dark cycles at 28° C.). The green portions of approximately 10 seedlings were cut using a scissors prior to stress treatments in vitro. To induce low-temperature stress, the seedlings were incubated at 4° C. in water for up to 6 h under continuous 150 μmol m⁻²s⁻¹ light. For high-salinity stress treatments, they were incubated in 400 mM NaCl for 2 h at 28° C. under continuous 150 μmol m⁻²s⁻¹ and to simulate drought stress they were air-dried for 2 h at 28° C. under continuous 150 μmol m⁻²s⁻¹ light. The Fv/Fm values were then measured as previously described (Oh et al., (2005) Plant Physiol 138: 341-351).

Rice 3′-Tiling Microarray Analysis

Expression profiling was conducted using the Rice 3′-Tiling Microarray, manufactured by NimbleGen Inc. (http://www.nimblegen.com/), which contains 27,448 genes deposited at the IRGSP, RAP1 database (http://rapdb.lab.nig.ac.jp). Further information on this microarray including statistical analysis can be found at http://www.ggbio.com (GreenGene Biotech). Among the genes on the microarray, 20,507 are from representative RAP1 sequences with cDNA/EST supports and 6,941 genes have been predicted without cDNA/EST supports. Ten 60-nt long probes were designed from each gene starting at 60 bp ahead of the stop codon and with 10 bp shifts in position so that 10 probes covered 150 bp within the 3′ region of the gene. In total, 270,000 probes were designed in this way (average size, 60-nt) to have Tm values of between 75° C. and 85° C. Random GC probes (38,000) were used to monitor the hybridization efficiency and fiducial markers at the four corners (225) were included to assist with overlaying of the grid on the image.

To identify stress-inducible AP2 genes in rice, total RNA (100 μg) was prepared using 14-d-old rice leaves from plants subjected to drought, high-salinity, ABA, and low-temperature stress conditions. For the high salinity and ABA treatments, the 14-d-old seedlings were transferred to a nutrient solution containing 400 mM NaCl or 100 μM ABA for 2 h in the greenhouse under continuous light of approximately 1000 μmol m⁻² s⁻¹. For drought treatment, 14-d-old seedlings were air-dried for 2 h under continuous light of approximately 1000 μmol m⁻² s⁻¹. For low temperature treatments, 14-d-old seedlings were exposed at 4° C. in a cold chamber for 6 h under continuous light of 150 μmol m⁻² s⁻¹.

For identification of genes up-regulated in OsCc1:AP37, OsCa1:AP59 plants, total RNA (100 μg) was prepared from leaf tissues of 14-d-old transgenic and nontransgenic rice (Oryza sativa cv Nakdong) seedlings grown under normal growth conditions. The mRNA was purified using the Qiagen oligotex column (Qiagen, Valencia, Calif.), according to the manufacturer's instructions. For normalization, data were processed with cubic alpine normalization using quartiles to adjust signal variation between chips, and with Robust Multi-Chip Analysis using a median polish algorithm implemented in NimbleScan (Workman et al., (2002) Genome Biol 3: research0048.1-research0048.16; Irizarry et al., (2003) Nucleic Acids Res 31: No. 4 e15). To assess the reproducibility of the microarray analysis, we repeated the experiments three times with independently prepared total RNAs and analyzed statistically of each data set using one-way ANOVA.

RT-PCR and qPCR Analysis

Total RNA was prepared as previously reported (Oh et al., (2008) Plant Biotechnol Rep 2: 227-231). For the analysis of target by RT-PCR, a cDNA synthesis system (Invitrogen, Carlsbad, Calif.) was used according to the manufacturer's instructions. PCR products were amplified using primers designed with Primer Designer 4 software (Sci-ed Software, Durham, N.C.). RT-PCR was carried out using the primer pairs at a final concentration of 10 pM each and 2 μL (equivalent to 5 ng total RNA) of cDNA as the template. PCR was performed at 95° C. for 10 mM, followed by 20 to 25 cycles of at 94° C. for 30 s, 57° C. for 30 s and 68° C. for 1 min. Amplified products were resolved on a 2% agarose gel. To validate our RT-PCR results, we repeated each experiment twice with independently prepared total RNA.

For quantitative real-time PCR experiments, SuperScript™ III Platinum®One-Step Quantitative RT-PCR system (Invitrogen, Carlsbad, Calif.) was used. For PCR reactions, a master mix of reaction components was prepared, according to the manufacturer's protocol for Platinum®SYBR®Green qPCR SuperMix-UDG (Invitrogen, Carlsbad, Calif.). Thermocycling and fluorescence detection were performed using a Stratagene Mx3000p Real-Time PCR machine (Stratagene, La Jolla, Calif.). PCR was performed at 95° C. for 10 min, followed by 20 to 25 cycles of at 94° C. for 30 s, 57° C. for 30 s and 68° C. or 1 min. To validate our qPCR results, we repeated each experiment three times.

Drought Treatments in the Field for Reproductive Stage Rice Plants

To evaluate yield components of transgenic plants under normal field conditions, three independent T₅ homozygous lines of the OsCc1:AP37and OsCc1:AP59 plants, together with nontransgenic (NT) controls were transplanted to a paddy field at the RDA, Suwon, Korea. A completely randomized design was employed with two replicates, each consisting of 4 plots of 5 m². At 25 d after sowing, the seedlings were randomly transplanted within a 15×30 cm distance, Fertilizer was applied at 70N/40P/70K kg/ha after the last paddling and 45 d after transplantation. Yield parameters were scored for 10 plants per plot, and 20 plants per line.

To evaluate yield components of transgenic plants under drought field conditions, three independent T₅ homozygous lines of the OsCc1:AP37and OsCc1:AP59 plants, and NT controls were transplanted to a removable rain-off shelter with a 1 meter deep container filled with natural paddy soil and located at Myongji University, Yongin, Korea. A completely randomized design, transplanting distance and use of fertilizer was employed as described above for normal field conditions. Drought stress was applied at the panicle heading stage (from 10-d before heading to 20-d after heading) by flowing water through a drain at the bottom of the container. To prevent plants from dying, we irrigated twice when the plant leaves rolled during drought stress. After exposure to drought stress conditions, the polyvinyl roofs were removed and plants were irrigated until harvesting.

When the plants grown under normal and drought conditions had reached maturity and the grains had ripened, they were harvested and threshed by hand (separation of seeds from the vegetative parts). The unfilled and filled grains were taken apart, independently counted using a Countmate MC1000H (Prince Ltd, Korea), and weighed. The following agronomic traits were scored: flowering date, panicle length, number of tillers, number of panicles, spikelets per panicle, filling rate (%), total grain weight (g), and 1,000 grain weight (g). The results from three independent lines were separately analyzed by one way ANOVA and compared with those of the NT controls. The ANOVA was used to reject the null hypothesis of equal means of transgenic lines and NT controls (P<0.05). The SPSS version 16.0 was used to perform statistical analysis.

### EXAMPLE 1

### Identification of Stress-Inducible APETELA2 Transcription Factors in Rice

Previously, the rice genome was predicted to contain 139 APETELA2 (AP2)-domain genes (Nakano et al., (2006) Plant Physiol 140: 411-432). To identify stress-inducible AP2 genes, we performed expression profiling with the Rice 3′-Tiling microarray (GreenGene Biotech, Yongin, Korea) using RNAs from 14-d-old rice leaves of seedlings subjected to drought, high-salinity, ABA, and low-temperature. When three replicates were averaged and compared with untreated leaves, a total of 42 genes were found to be up-regulated by 1.6-fold or greater (P<0.05) by one or more stress conditions including drought, high-salinity, ABA, and low-temperature (Table 1).

TABLE 1
Rice APETALA2 Transcription Factor Genes Up-Regulated Under Stress
Conditions.

| | | | | | | | | Low | |
|---|---|---|---|---|---|---|---|---|---|
| | | Drought | | High Salinity | | ABA | | temperature | |
| Subgroups | SEQ. ID^{a} | Mean^{b} | Pval^{c} | Mean^{b} | Pval^{c} | Mean^{b} | Pval^{c} | Mean^{b} | Pval^{c} |
| I | AP37 | 3.8 | 0.00 | 3.4 | 0.00 | 1.1 | 0.72 | 1.9 | 0.10 |
| | Os04g0610400 | 2.1 | 0.01 | 1.5 | 0.17 | −1.2 | 0.65 | 2.9 | 0.02 |
| | AK067373 | 2.8 | 0.01 | 2.6 | 0.02 | 1.3 | 0.69 | 2.9 | 0.04 |
| | AK111755 | 2.7 | 0.00 | 1.8 | 0.05 | 1.1 | 0.86 | 2.5 | 0.04 |
| II | AP59 | 7.7 | 0.00 | 6.1 | 0.00 | 1.3 | 0.43 | 8.6 | 0.00 |
| | AY339378 | 1.4 | 0.27 | 2.8 | 0.01 | 1.2 | 0.83 | −1.1 | 0.91 |
| | AY341826 | 1.3 | 0.27 | 1.1 | 0.92 | 1.3 | 0.52 | 10.1 | 0.00 |
| | AK109390 | 3.6 | 0.00 | 2.4 | 0.02 | −1.4 | 0.39 | 1.2 | 0.72 |
| III | Os03g0191900 | 12.7 | 0.00 | 4.9 | 0.00 | −1.3 | 0.42 | 1.2 | 0.68 |
| | Os05g0572000 | 3.5 | 0.00 | 3.2 | 0.00 | 1.4 | 0.29 | −1.1 | 0.87 |
| | AK063886 | 1.8 | 0.03 | 1.2 | 0.58 | −1.6 | 0.13 | −1.3 | 0.43 |
| | AK068312 | 9.1 | 0.00 | 5.2 | 0.00 | 1.0 | 0.98 | 1.2 | 0.61 |
| IV | AK111775 | 1.5 | 0.29 | 1.3 | 0.69 | 3.2 | 0.02 | 4.2 | 0.03 |
| | AK103783 | 1.2 | 0.41 | 1.5 | 0.14 | 1.9 | 0.04 | −1.0 | 0.93 |
| | AK101949 | 2.0 | 0.01 | 2.6 | 0.00 | 12.8 | 0.00 | 1.4 | 0.30 |
| | AK069833 | 1.8 | 0.04 | 1.7 | 0.04 | 1.6 | 0.17 | −1.3 | 0.51 |
| | AK109380 | 2.3 | 0.01 | 2.7 | 0.01 | 4.0 | 0.00 | 2.1 | 0.07 |
| | AY297447 | 1.3 | 0.33 | 1.8 | 0.03 | −1.0 | 0.96 | 1.8 | 0.09 |
| | AK106057 | −1.9 | 0.19 | −1.2 | 0.89 | 22.0 | 0.00 | 2.0 | 0.30 |
| | AK060090 | 1.2 | 0.55 | −1.0 | 0.99 | 2.3 | 0.02 | 1.2 | 0.69 |
| V | OsDREB1A | 2.4 | 0.01 | −1.1 | 0.91 | −1.8 | 0.07 | 7.7 | 0.00 |
| | OsDREB1B | 9.5 | 0.00 | 6.0 | 0.00 | 1.2 | 0.67 | 3.1 | 0.04 |
| | AY345234 | 6.3 | 0.00 | 5.7 | 0.00 | −1.3 | 0.62 | 7.9 | 0.00 |
| | Os08g0545500 | 2.2 | 0.01 | 1.5 | 0.24 | −1.0 | 0.99 | 1.5 | 0.33 |
| | AK106041 | 1.6 | 0.19 | 1.1 | 0.95 | 1.0 | 0.98 | 8.2 | 0.00 |
| | AF300972 | −1.2 | 0.40 | −1.9 | 0.02 | −3.2 | 0.00 | 51.4 | 0.00 |
| | Os09g0522100 | 1.6 | 0.17 | −1.1 | 0.93 | 1.1 | 0.90 | 6.4 | 0.01 |
| VI | OsDREB2A | 3.0 | 0.01 | 3.8 | 0.01 | 2.2 | 0.07 | −1.6 | 0.41 |
| | AK071850 | 2.9 | 0.00 | 2.2 | 0.00 | 1.5 | 0.09 | 1.2 | 0.50 |
| Others | AK107775 | 2.8 | 0.00 | 2.6 | 0.01 | 1.8 | 0.05 | 1.1 | 0.83 |
| | Os02g0797100 | 3.3 | 0.01 | 1.6 | 0.37 | −1.0 | 0.95 | 1.6 | 0.48 |
| | AK101133 | 1.8 | 0.01 | 1.2 | 0.63 | 1.1 | 0.70 | 1.0 | 0.99 |
| | AK100575 | 1.2 | 0.43 | 1.5 | 0.12 | 2.1 | 0.01 | −1.0 | 0.98 |
| | Os01g0888000 | 2.8 | 0.02 | 1.8 | 0.18 | 1.6 | 0.30 | 1.7 | 0.32 |
| | AK107852 | 9.7 | 0.01 | 3.9 | 0.07 | 1.4 | 0.75 | 2.3 | 0.38 |
| | AK101501 | 2.0 | 0.01 | 2.3 | 0.01 | 1.5 | 0.17 | −1.0 | 0.99 |
| | AK107125 | 5.1 | 0.00 | 3.9 | 0.00 | 1.7 | 0.26 | 1.3 | 0.67 |
| | AK107146 | 9.4 | 0.00 | 4.2 | 0.00 | −1.0 | 0.96 | 6.9 | 0.01 |
| | AK062882 | 21.2 | 0.00 | 14.1 | 0.00 | 2.2 | 0.05 | 26.7 | 0.00 |
| | AK067195 | 15.2 | 0.00 | 7.7 | 0.00 | 1.2 | 0.67 | 62.5 | 0.00 |
| | AY341827 | 3.1 | 0.00 | 3.2 | 0.01 | 5.1 | 0.00 | −1.2 | 0.65 |
| | AK108208 | 4.1 | 0.00 | 3.0 | 0.01 | −1.1 | 0.85 | −1.1 | 0.84 |

Numbers in bold indicate up-regulation by more than 1.6-fold (P < 0.05) in plants grown under stress conditions.
^{a}Numbers for full-length cDNA sequences of the corresponding genes.
^{b}Numbers represent the mean of three independent biological replicates. These microarray data sets can be found at http://www.ncbi.nlm.nih.gov/geo/(Gene Expression Omnibus, GEO).
^{c}P values were analyzed by one-way ANOVA.

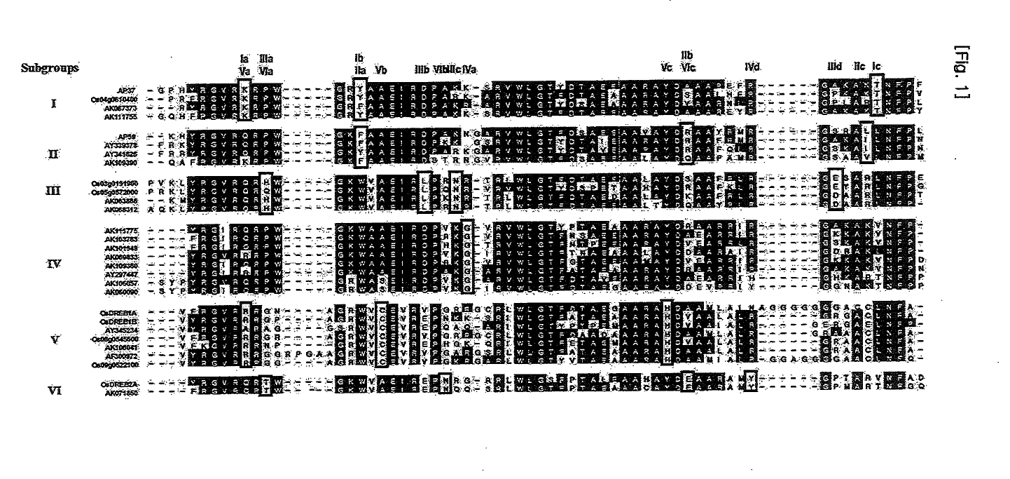

Phylogenic analysis of the amino acid sequences of 42 factors revealed the presence of 6 subgroups (I to VI), with AP37 assigned to subgroup I, AP59 to subgroup II, OsDREB1A to subgroup V, and OsDREB2A to subgroup VI (FIG. 1, Dubouzet et al., (2003) Plant J 33: 751-763). Thirteen out of 42 factors are not classified into any of the 6 subgroups. Comparison of the amino acid sequence spanning the AP2 domain identified signature motifs by which these subgroups can be distinguished (FIG. 1). For example, signature motifs Ia, Ic, IIb, and IIc are specific to subgroups I and II, respectively, and motif Ib (IIa) is common to both. In addition to sequence similarity, members of each subgroup are closely related in terms of their response to stress. For example, the expression of the genes in subgroups I, II and V is not induced by ABA; whereas, that of the members of subgroups III and VI is not induced by ABA nor by low temperature.

Two genes, AP37 (AK061380) and AP59 (AK073812), representing subgroups I and II, respectively, were functionally characterized in our current study. The transcript levels of AP37 and AP59 were measured by RNA gel-blot analysis using total RNAs from leaf tissues of 14-d-old seedlings exposed to high salinity, drought, ABA and low temperature (FIG. 2). The expression of both AP37 and AP59 was found to be induced after 2 hours of exposure to high salinity and drought stress. AP37 differs from AP59 in its response to low temperature and ABA. The expression of the former responded rapidly to low temperature and was induced by ABA, whereas, the latter responded slowly to low temperature and was not induced by ABA. This is somewhat inconsistent with the microarray results, which indicated that AP37 is not induced by ABA. This discrepancy may be due to variation in the stress treatments. Overall, our results showed that AP37 and AP59 are stress-inducible AP2 genes that are closely related, yet different in their expression profiles.

### EXAMPLE 2

### Stress Tolerance of OsCc1:AP37 and OsCc1:AP59 Plants at the Vegetative Stage

To enable the overexpression of the AP37 and AP59 genes in rice, their full-length cDNAs were isolated and linked to the OsCc1 promoter for constitutive expression, generating the constructs OsCc1:AP37 and OsCc1:AP59 (FIG. 3). These constructs were then introduced into rice by Agrobacterium-mediated transformation, which yielded 15 to 20 independent transgenic lines per construct. Transgenic T₁₋₄ seeds were collected and three independent T₄₋₅ homozygous lines of both OsCc1:AP37 and OsCc1:AP59 plants were selected for further analysis. All of the transgenic lines grew normally with no stunting. The transcript levels of AP37 and AP59 in the OsCc1:AP37 and OsCc1:AP59 plants were determined by RNA-gel blot analysis. For this purpose, total RNAs were extracted from leaf tissues of 14-d-old seedlings grown under normal growth conditions (FIG. 4). Transcript levels of AP37 and AP59 were clearly enhanced at various levels in different transgenic lines as compared to those in the nontransgenic (NT) controls. To investigate whether the overexpression of AP37 and AP59 correlated with stress tolerance in rice, four-week-old transgenic plants and NT controls were exposed to drought stress (FIG. 5). The NT plants started to show visual symptoms of drought-induced damage, such as leaf rolling and wilting with a concomitant loss of chlorophylls, at an earlier stage than the OsCc1:AP37 and OsCc1:AP59 plants. The transgenic plants also recovered faster than the NT plants upon re-watering. Consequently, the NT plants remained severely affected by the time at which all of the transgenic lines had fully recovered except for the OsCc1:AP37-9 line. This line recovered slower than the other transgenic lines, probably due to a lower level of transgene expression than others (FIGS. 4 and 5). Levels of transgene expression in the OsCc1:AP59-2 line is also lower than those of others while its phenotype is comparable to others (FIGS. 4 and 5). Thus, the difference in transgenic phenotype does not always reflect the different levels of mRNA.

To further verify the stress-tolerance phenotype, we measured the Fv/Fm values of the transgenic and NT control plants, all at the vegetative stage (FIG. 6). The Fv/Fm values represent the maximum photochemical efficiency of PS II in a dark-adapted state (Fv, variable fluorescence; Fm, maximum fluorescence). The Fv/Fm levels were about 30% and 20% higher in the OsCc1:AP37 and OsCc1:AP59 plants, respectively, than in the NT plants under drought and high salinity conditions. Under low temperature conditions, in contrast, the Fv/Fm levels were 15% higher in the OsCc1:AP37 plants than in the NT plants whereas the levels in the OsCc1:AP59 plants were at similar levels to those of the NT plants. Hence, our results indicate that the overexpression of AP37 and AP59 in transgenic rice increases the tolerance of these plants to drought and high salinity, stress conditions during the vegetative stage but that an increased tolerance to low temperature occurs only in plants overexpressing AP37.

### EXAMPLE 3

### Identification of Genes Up-Regulated by Overexpressed AP37 and AP59

To identify genes that are up-regulated by the overexpression of AP37 and AP59, we performed expression profiling of OsCc1:AP37 and OsCc1:AP59 plants in comparison with NT controls under normal growth conditions. Expression profiling with the Rice 3′-Tiling microarray was conducted using RNA samples extracted from 14-d-old leaves of these transgenic plants and NT controls, all grown under normal growth conditions. Each data set was obtained from three biological replicates. Statistical analysis of each data set using one-way ANOVA identified 85 genes that are up-regulated by AP37 and/or AP59 with 3-fold or greater induction in the transgenic plants than in NT plants (P<0.05). Specifically, a total of 37 genes were found to be commonly activated by AP37 and AP59 whereas 10 and 38 genes are specific to AP37 and AP59, respectively. Based on our microarray data shown in Tables 1, we selected 8 stress-inducible genes out of 37 common target genes and verified their AP37- and AP59-dependent expression patterns under normal growth conditions by RT-PCR (FIG. 7, control). To test whether their expression levels increase further under stress conditions, we measured the transcript levels of the 8 genes in OsCc1:AP37, OsCc1:AP59, and NT plants after exposure to drought, high salinity and low temperature conditions (FIGS. 7 and 8). In NT plants, all of the target genes were induced at various levels within 2 hours of the stress treatments. In the transgenic plants, in contrast, the transcripts levels of many genes are lower under stress conditions in comparison with stress-treated NT plants. The same is true for the AP59 gene in OsCc1:AP37 plants exposed to drought, high salinity, and low temperature conditions, and also for AP37 in OsCc1:AP59 plants grown under low temperature conditions. Such lower levels of target gene transcripts in OsCc1:AP37 and OsCc1:AP59 plants than in NT plants under stress conditions were not unexpected because the transgenic plants were less affected by stress damage compared with the NT controls. The transgenic plants were more tolerant to stress than the NT controls at the time of stress treatments, hence stress-induced expression levels of target genes being smaller in the transgenic plants than in NT controls. We repeated the experiments shown in FIG. 7 by using real-time PCR, obtaining results similar to those of RT-PCR (FIG. 8). Overall, our results suggest that the AP37 and AP59 genes enhance stress tolerance differently by activating distinct groups of stress-regulated genes.

### EXAMPLE 4

### Overexpression of AP37 Increases Rice Grain Yield Under Drought Conditions

A phenotypic evaluation of both OsCc1:AP37, OsCc1:AP59 and NT control plants revealed no major differences in the vegetative growth of the entire plants. To then investigate whether the overexpression of AP37 and AP59 improved the rice grain yield of transgenic plants under field conditions, we transplanted three independent T₅ homozygous lines of the OsCc1:AP37and OsCc1:AP59 plants, together with their respective nontransgenic (NT) controls, to a paddy field and grew them to maturity. A completely randomized design with two replicates was employed. The subsequent evaluation of the yield parameters of these plants revealed that the grain yield of OsCc1:AP37 plants remained similar to that of the NT controls under normal field conditions (FIG. 9 and Table 2).

TABLE 2
Analysis of Seed Production Parameters in OsCc1:AP37 and OsCc1:AP59
Plants Under Normal Growth Conditions. Each parameter represents the mean ± SD
(n = 20) for OsCc1:AP37 and OsCc1:AP59, and the respective nontransgenic (NT)
control plants. The percentage differences (%Δ) between the values for the
OsCc1:AP37 or OsCc1:AP59 plants and for the respective NT controls were
calculated. P values were determined according to the least significant difference
(LSD) test.

| | | Panicle length | No. of spikelets | Total No. of spikelets | Filling rate | No. of filled grain | Total grain weight |
|---|---|---|---|---|---|---|---|
| Constructs | | (cm) | (per panicle) | (per hill) | (%) | (per hill) | (g) |
| NT | | 18.32 ± 1.37 | 96.04 ± 12.74 | 1209.70 ± 137.55 | 90.80 ± 3.64 | 1098.45 ± 133.26 | 22.61 ± 3.03 |
| AP37-4 | | 19.70 ± 1.25 | 95.86 ± 8.19 | 1294.55 ± 244.75 | 91.23 ± 2.82 | 1177.65 ± 202.93 | 24.56 ± 5.28 |
| | % Δ | 7.53 | −0.19 | 7.01 | 0.47 | 7.21 | 9.11 |
| | P value | 0.011 | 0.951 | 0.151 | 0.886 | 0.144 | 0.129 |
| AP37-5 | | 18.17 ± 1.18 | 90.78 ± 9.50 | 1138.95 ± 152.68 | 87.15 ± 3.60 | 992.80 ± 161.39 | 21.60 ± 3.83 |
| | % Δ | −0.82 | −5.50 | −0.01 | −4.02 | −9.62 | −4.04 |
| | P value | 0.776 | 0.086 | 0.218 | 0.685 | 0.053 | 0.500 |
| AP37-9 | | 19.10 ± 2.48 | 83.68 ± 7.10 | 1205.95 ± 188.49 | 88.24 ± 2.86 | 1066.50 ± 162.78 | 22.31 ± 4.39 |
| | % Δ | 4.26 | −12.87 | −0.31 | −2.82 | −2.91 | −0.89 |
| | P value | 0.144 | 0.000 | 0.949 | 0.076 | 0.553 | 0.884 |
| NT | | 18.80 ± 1.19 | 98.90 ± 6.99 | 1327.10 ± 191.16 | 92.84 ± 3.01 | 1232.55 ± 185.73 | 25.78 ± 4.39 |
| AP59-1 | | 16.95 ± 1.14 | 71.38 ± 11.78 | 996.10 ± 181.07 | 73.90 ± 7.57 | 734.75 ± 158.01 | 14.72 ± 4.34 |
| | % Δ | −9.84 | −27.81 | −24.04 | −20.39 | −40.38 | −42.88 |
| | P value | 0.000 | 0.000 | 0.000 | 0.000 | 0.000 | 0.000 |
| AP59-2 | | 18.60 ± 1.50 | 71.70 ± 5.37 | 1044.20 ± 241.54 | 78.30 ± 6.08 | 811.20 ± 167.09 | 17.97 ± 4.42 |
| | % Δ | −1.06 | −27.50 | −21.31 | −15.86 | −34.18 | −30.27 |
| | P value | 0.622 | 0.000 | 0.000 | 0.000 | 0.000 | 0.000 |
| AP59-6 | | 18.45 ± 1.23 | 80.13 ± 10.63 | 1045.10 ± 188.85 | 88.01 ± 3.60 | 919.25 ± 165.62 | 19.70 ± 4.19 |
| | % Δ | −1.87 | −18.97 | −21.24 | −5.19 | −25.41 | −23.68 |
| | P value | 0.389 | 0.000 | 0.000 | 0.006 | 0.000 | 0.000 |

In the OsCc1:AP59 plants under the same field conditions however, total grain weight was reduced by 23-43% compared with the NT controls, which appears to be due to decreases in the number of spikelets. These observations prompted us to examine the yield components of the transgenic rice plants grown in the field under drought conditions. Three independent lines of the OsCc1:AP37and OsCc1:AP59 plants, and NT controls were transplanted to a paddy field with a removable rain-off shelter, and exposed to drought stress at the panicle heading stage (from 10-d before heading to 20-d after heading). Statistical analysis of the yield parameters showed that the decrease in grain yield under drought conditions was significantly smaller in the OsCc1:AP37 plants than that observed in the controls. Specifically, in the drought-treated OsCc1:AP37 plants, the filling rate was higher than the drought-treated NT plants by 17-36%, which resulted in an increase in the total grain weight by 16-57%, depending on the transgenic line (FIG. 9 and Table 3).

TABLE 3
Analysis of Seed Production Parameters in OsCc1:AP37 and OsCc1:AP59
Plants Under Drought Stress Conditions. Each parameter represents the mean ± SD
(n = 20) for OsCc1:AP37 and OsCc1:AP59, and the respective nontransgenic (NT)
control plants. The percentage differences (%Δ) between the values for the
OsCc1:AP37 or OsCc1:AP59 plants and for the respective NT controls were
calculated. P values were determined according to the least significant difference
(LSD) test.

| | | Panicle length | No. of spikelets | Total No. of spikelets | Filling rate | No. of filled grain | Total grain weight |
|---|---|---|---|---|---|---|---|
| Constructs | | (cm) | (per panicle) | (per hill) | (%) | (per hill) | (g) |
| NT | | 19.50 ± 2.38 | 98.34 ± 14.52 | 1526.76 ± 753.86 | 60.15 ± 16.39 | 871.75 ± 349.09 | 15.20 ± 7.29 |
| AP37-4 | | 19.10 ± 1.51 | 92.49 ± 11.71 | 1443.80 ± 682.61 | 82.24 ± 10.53 | 1144.40 ± 472.48 | 20.60 ± 9.07 |
| | % Δ | −2.05 | −5.94 | −5.43 | 36.71 | 31.27 | 35.52 |
| | P value | 0.703 | 0.458 | 0.819 | 0.020 | 0.261 | 0.273 |
| AP37-5 | | 17.60 ± 0.91 | 93.91 ± 6.73 | 1741.25 ± 212.78 | 72.88 ± 10.67 | 1282.50 ± 203.78 | 23.87 ± 3.75 |
| | % Δ | −10.25 | 4.60 | 14.04 | 21.15 | 44.62 | 57.07 |
| | P value | 0.087 | 0.592 | 0.578 | 0.171 | 0.134 | 0.105 |
| AP37-9 | | 18.85 ± 0.75 | 87.29 ± 11.10 | 1483.75 ± 106.22 | 70.92 ± 11.68 | 1059.75 ± 240.49 | 17.70 ± 4.76 |
| | % Δ | −13.48 | −11.23 | −2.81 | 17.89 | 21.56 | 16.44 |
| | P value | 0.030 | 0.194 | 0.910 | 0.242 | 0.455 | 0.624 |
| NT | Mean | 19.75 ± 2.08 | 97.01 ± 12.27 | 1638.25 ± 737.31 | 60.31 ± 16.38 | 895.25 ± 307.00 | 15.45 ± 6.82 |
| AP59-1 | | 15.37 ± 2.49 | 64.86 ± 20.71 | 1001.25 ± 689.62 | 63.20 ± 10.24 | 663.75 ± 481.75 | 9.93 ± 10.41 |
| | % Δ | −22.15 | −43.45 | −34.90 | 4.78 | −25.85 | −35.76 |
| | P value | 0.008 | 0.030 | 0.199 | 0.781 | 0.358 | 0.324 |
| AP59-2 | | 18.37 ± 0.94 | 92.25 ± 37.81 | 1133.75 ± 431.63 | 53.66 ± 15.71 | 579.25 ± 170.12 | 10.40 ± 4.35 |
| | % Δ | −6.96 | −4.89 | −28.29 | −11.01 | −36.29 | −32.68 |
| | P value | 0.331 | 0.785 | 0.325 | 0.626 | 0.215 | 0.366 |
| AP59-6 | | 17.17 ± 1.78 | 60.89 ± 12.68 | 1137.33 ± 280.32 | 78.88 ± 14.19 | 902.33 ± 317.88 | 16.37 ± 7.36 |
| | % Δ | −13.08 | −31.23 | −26.06 | 30.76 | 0.79 | −0.53 |
| | P value | 0.105 | 0.076 | 0.365 | 0.119 | 0.979 | 0.889 |

In the drought-treated OsCc1:AP59 plants, in contrast, the total grain weight was reduced by about 30% when compared with the drought-treated NT controls, which was similar to the yields observed for these plants under normal growth conditions. Despite the similar levels of drought tolerance at the vegetative stage of the OsCc1:AP37and OsCc1:AP59 plants, a sharp difference in grain yield under drought conditions is actually not surprising. This is because, unlike in OsCc1:AP37 plants, spikelet development of OsCc1:AP59 plants were significantly affected by the constitutive overexpression of AP59 under both normal and drought conditions, which resulted in a loss of grain yield. Taken together, our current results suggest that the overexpression of AP37 confers drought tolerance in rice at the reproductive stage and improves grain yield significantly.

1. A transcription factor protein AP37 (APETELA 37) originating from rice, which consists of an amino acid sequence of SEQ ID NO: 2.

2. A gene which encodes the AP37 protein of claim 1.

3. The gene according to claim 1, characterized in that the gene consists of a nucleotide sequence of SEQ ID NO: 1.

4. A recombinant vector comprising the gene of claim 2.

5. The host cell transformed with the recombinant vector of claim 4.

6. The host cell according to claim 5, characterized in that it is a plant cell.

7. A method of producing a transgenic plant comprising steps of:
transforming a plant cell with the recombinant vector of claim 4; and
regenerating a transgenic plant from the transformed plant cell.

8. A method of improving tolerance of a plant to stress comprising a step of transforming a plant cell with the recombinant vector of claim 4 to overexpress the AP37 gene in the plant.

9. The method according to claim 8, characterized in that the stress is drought, high salinity or low temperature stress.

10. A method of increasing grain yield comprising a step of transforming a plant cell with the recombinant vector of claim 4 to overexpress the AP37 gene in the plant under drought condition.

11. A plant having improved tolerance to stress which is produced according to the method of claim 8.

12. A plant having increased grain yield which is produced according to the method of claim 10.

13. The plant according to claim 11, characterized in that the plant is rice.

14. The plant according to claim 12, characterized in that the plant is rice.

15. A composition for improving tolerance of a plant to stress comprising as an effective component the gene encoding AP37 protein, which consists of the amino acid sequence of SEQ ID NO: 2.

16. A composition for increasing grain yield under drought condition comprising as an effective component the gene encoding AP37 protein, which consists of the amino acid sequence of SEQ ID NO: 2.
